(19) United States
(12) Patent Application Publication
(10) Pub. No.: US 2015/0258148 A1
(43) Pub. Date: Sep. 17, 2015

(54) Compositions, Methods and Systems for Cellular Differentiation from Stem Cells

(71) Applicant: Kiminobu Sugaya, Winter Park, FL (US)

(72) Inventor: Kiminobu Sugaya, Winter Park, FL (US)

(73) Assignee: University of Central Florida Research Foundation, Inc., Orlando, FL (US)

(21) Appl. No.: 14/404,744

(22) PCT Filed: Jun. 6, 2013
(86) PCT No.: PCT/US13/44599

§ 371 Date: Dec. 1, 2014

Related U.S. Application Data
(60) Provisional application No. 61/658,640, filed on Jun. 12, 2012.
(60) Provisional application No. 61/656,485, filed on Jun. 6, 2012.

(51) Int. Cl. A61K 35/35; C12N 5/077

(52) U.S. Cl. CPC A61K 35/35; C12N 5/0653; C12N 2506/21; C12N 2501/155

(57) ABSTRACT

The present invention is directed to methods and systems directed to altering the differentiation of a cell, more particularly to biasing a potent cell, such as a mesenchymal stem cell, by contacting such cell with a protein having properties of bone morphogenic protein-4. The contacting may be achieved via genetic modification, and the resulting cell may be or have characteristics of a pre-adipocyte. Pre-adipocyte cells so-obtained, and their progeny, may be used for various purposes, including in cosmetic and other surgical therapies and treatments.

### CROSS REFERENCE TO RELATED APPLICATIONS

This application is related to U.S. Provisional Application No. 61/658,640 filed Jun. 12, 2012 and U.S. Provisional Application No. 61/656,485 filed Jun. 6, 2012, to which priority is claimed under 35 USC 119.

### STATEMENT REGARDING SEQUENCE LISTING

Sequences of nucleotides and/or proteins may be provided herein, with relevant source information and/or references to the same. Applicant reserves the right to present any such sequences in a formal sequence listing at a later date, such as in a non-provisional patent application claiming priority to this application.

### BACKGROUND

Proper cellular function and differentiation depends on intrinsic signals and extracellular environmental cues. These signals and cues vary over time and location in a developing organism (i.e., during embryogenesis), and remain important in developing and differentiating cells during post-natal growth and in a mature adult organism. Thus, in a general sense, the interplay of the dynamically changing set of intracellular dynamics (such as manifested by intrinsic chemical signaling and control of gene expression) and environmental influences (such as signals from adjacent cells) determine cellular activity. The cellular activity so determined is known to include cell migration, cell differentiation, and the manner a cell interacts with surrounding cells.

The use of stem cells and stem-cell-like cells of various types for cell replacement therapies, and for other cell-introduction-based therapies, is being actively pursued by a number of researchers. Embryonic stems cells from a blastocyst stage are frequently touted for their pluripotency—that is, their ability to differentiate into all cell types of the developing organism. Later-stage embryonic stem cells, and certain cells from generative areas of an adult organism, are identified as more specialized, multipotent stem cells. These cells include cells that are able to give rise to a succession of a more limited subset of mature end-stage differentiated cells of particular types or categories, such as hematopoietic and mesenchymal stem cells.

### SUMMARY OF THE INVENTION

The invention may also be said broadly to consist in the parts, elements and features referred to or indicated in the specification of the application, individually or collectively, in any or all combinations of two or more of said parts, elements or features, and where specific integers are mentioned herein which have known equivalents in the art to which the invention relates, such known equivalents are deemed to be incorporated herein as if individually set forth.

### BRIEF DESCRIPTION OF THE DRAWINGS

FIG. 1A-D shows an Oil Red O staining comparison of differentiation Media A and Media B in both ADSCs and MSCs. (A) ADSCs exposed to Media A. (B) ADSCs exposed to Media B. (C) MSCs exposed to Media A. (D) MSCs exposed to Media B.

FIG. 2A-D shows Oil Red O staining confirming the presence of lipid droplets in both the ADSCs and MSCs. (A) ADSCs without staining. (B) ADSCs positive staining for lipid droplets. (C) MSCs without staining. (D) MSCs positive staining for lipid droplets.

FIG. 3 A, B provides images demonstrating immunocytochemistry results of ADSCs (A) and MSCs (B) cultured in the presence of BMP4 showed strong immunoreaction for the DLK-1 (Pref-1) protein, characteristic of PAs. There was, however, no expression of the AP-2 protein characteristic of adipocytes.

FIG. 4A-D Flow Cytometry Analysis of ADSCs and MSCs (A) Negative α-actin staining of ADSCs. (B) Positive Pref-1 staining of MSCs. (C) Positive α-actin staining of MSCs. (D) Positive Pref-1 staining of MSCs.

### DETAILED DESCRIPTION

In reviewing the detailed disclosure which follows, and the specification more generally, it should be borne in mind that all patents, patent applications, patent publications, technical publications, scientific publications, and other references referenced herein are hereby incorporated by reference in this application to the extent they are not inconsistent with the teachings herein.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed.

It is important to an understanding of the present invention to note that all technical and scientific terms used herein, unless defined herein, are intended to have the same meaning as commonly understood by one of ordinary skill in the art. The techniques employed herein are also those that are known to one of ordinary skill in the arartart, unless stated otherwise. For purposes of more clearly facilitating an understanding the invention as disclosed and claimed herein, the following definitions are provided.

Though methods of biasing the differentiation of potent cells (and including multipotent cells such as hematopoietic and mesenchymal stem cells) through the manipulation of environmental conditions in tissue culture are well characterized, such methods do not provide an implantable cell that maintains a desired level of potency to properly migrate and integrate to the tissue surrounding the implantation site. Thus, a method of biasing potent and multipotent cells prior to implantation to differentiate into a desired cell type after implantation is desired. Such biasing would provide for an improved percentage of such potent cells in a culture vessel or therapeutic composition, dosage or treatment to differentiate to this desired cell type. Improvements to the percentage of cells that are known to be biased to differentiate to desired cell types will enable improvements both in research and treatment technologies, including methods for treatment of diseases and other conditions of a subject in need thereof.

Thus, there is a need in the art to improve the compositions, methods and systems that provide biased and/or differentiated cells from stem cells or stem-cell-like cells. More particularly, a need exists to obtain a higher percentage of desired cells from a pre-implantation cell culture, such as starting from multipotent stem cells and obtaining a higher percentage of cells committed to differentiate to a specified type of functional nerve cell. The present invention addresses these needs.

As used herein, “isolated” means synthetic, or altered or removed from the natural state through human intervention.

As used herein, “under the control” of a promoter means that the nucleic acid sequences encoding the sense or antisense strands are located 3′ of the promoter, so that the promoter can initiate transcription of the sense or antisense coding sequences.

As used herein, a “subject” includes a human being or non-human animal. In various embodiments the subject is a human being.

As used herein, an “effective amount” is an amount sufficient to cause a desired therapeutic or other result.

Human mesenchymal stem cells (MSCs) are multipotent and found throughout the body. MSCs contribute to the renewal of tissues such as, bone, cartilage and fat (1). In order for these cells to be coaxed into one cell line, specific differentiation inducers are used. For example, 1-methyl-3-isobutylxanthine, dexamethasone, insulin, and indomethacin are used to force differentiation into the adipocyte lineage (2). There are also commercially available kits to commit MSCs into a variety of lineages. However, these kits are for research purposes only and are not available for clinical use. A recent study by Tang, et al. shows that adipose lineage commitment can be induced in murine embryonal cells, C3H10T½, by treatment with bone morphogenic protein-4 (BMP-4) alone (3). BMP-4 is a member of the TGF-β superfamily and acts on type 1a and 2 BMP receptors (4). Exposure of MSCs to BMP-4 directs them to the adipocyte lineage by differentiating the MSCs into pre-adipocytes (PAs). PAs are precursor cells to adipocytes, are fibroblast-like in nature and maintain the capacity for self-renewal (5). Their ability to self renew plays a central role in maintaining adipocyte populations in the body.

Human mesenchymal stem cells (MSCs) are found throughout the body and can be harvested from many sources including, blood, bone marrow, and adipose tissue. Their multipotency enables them to give rise to a variety of different lineages, such as, adipocytes, osteoblasts and chondrocytes. MSCs have been terminally differentiated, to the lineages mentioned above, using an array of techniques and commercially available kits. Current protocols, used to commit MSCs to different cell lineages, involve using a cocktail of differentiation inducers. For example, 3-isobutyl-1-methyl-xanthine, dexamethasone, insulin, and indomethacin are used to guide MSCs to the adipose lineage. A recent study shows that a commitment to the adipose lineage can be induced in murine embryonal cells by treatment with bone morphogenic protein-4 (BMP-4) only. Thus, it is believed that cells obtained from blood and adipose tissue treated with BMP-4 will also demonstrate commitment to the adipose lineage. Exposure of MSCs to BMP-4 directs them to the adipocyte lineage by differentiating the MSCs into pre-adipocytes (PAs). As noted, PAs are adipocyte precursor cells that are fibroblast-like in nature and maintain the capacity for self-renewal, and their ability to self renew plays a central role in maintaining adipocyte populations in the body.

The presence of intracellular lipid droplets, found in PAs and adipocytes, is a strong indicator a cell is from the adipose lineage. Oil Red O, a lysochrome diazo dye, is used to visualize the formation of lipid droplets within cells. Staining of BMP-4 treated cells obtained from blood and adipose tissue has been employed in the Example herein to confirm the presence of lipid droplets. The formation of lipid droplets confirms the ability of BMP-4 alone to commit cells obtained from blood or adipose tissue to the adipose lineage. However, both PAs and adipocytes contain lipid droplets. To determine if the BMP-4 treated cells were PAs and not adipocytes, they were tested for markers specific to PAs and not found in adipocytes. Antibody staining verified the expression of PA markers, and absence of adipocyte markers in the BMP-4 treated cells. Both staining methods prove that adipose commitment is possible in cells obtained from blood or adipose tissue with exposure to BMP-4 alone. The ability to create PAs derived from blood and adipose tissue will eliminate the need for using bone marrow, a very invasive method of collecting MSCs. Additionally, the self-renewal potential of PAs makes them a good alternative to lipoinjection therapies. Current therapies use lipoaspirates that are heterogeneous cell populations and contain low numbers of adipocyte precursor cells. A low population of PAs fails to establish an adipocyte regeneration pool, making the effects of lipoinjection short term. Increasing the population of PAs before injection, may increase the longevity of current lipoinjection procedures. Accordingly, one embodiment pertains to a method of isolating and/or identifying PAs as described above.

Thus, embodiments of the invention may include methods to obtain pre-adipocyte cells and their progeny cells, such as by treatment of blood or adipose tissue cells with bone morphogenic protein-4 (BMP-4) to obtain these cells and then adipocytes, compositions thereof, and compositions, methods, and systems for various uses of such cells, including but not limited to cosmetic and other surgical procedures wherein cells and compositions of such cells are utilized in a subject in need thereof, such as to fill, hide, or otherwise improve wrinkles, scars, depressions and other defects either externally (and internally as applicable), and/or for tissue augmentation, such as in breast augmentation, in said subject. The methods include use of such cells in lipoinjection.

To commit mesenchymal (or hematopoietic) stem cells to the adipose lineage using only bone morphogenic protein-4, various embodiments of the present invention are directed to the use of bone morphogenic protein-4 (BMP-4, also referred to as bone morphogenetic protein-4, and exemplified by recombinant human bone morphogenic protein-4 obtained from Invitrogen (Life Technologies, Carlsbad, Calif. USA, Catalog No. PHC9534), to differentiate (or bias) a stem cell to a pre-adipocyte, or alternatively to a cell having characteristics of a pre-adipocyte, such as being sufficient to be self-renewing (able to proliferate under culture conditions), able to induce angiogenesis, and able to develop into an adipocyte under suitable conditions. In various embodiments BMP-4 is the only additive provided for the purpose of committing/biasing such stem cells.

Moreover, it is noted that other cells, (for example, either stem cells of another type, less potent cells, such as adult somatic cells) may be manipulated through cell culture techniques so as to take on characteristics of MSCs, which can then in turn be biased toward becoming pre-adipocytes. Thus, the reference to mesenchymal stem cells or hematopoietic stem cells includes cells that may be of a sample of a natural source, whereby the natural source included MSCs or may be of a sample where cells in the sample were manipulated from their original state to become MSCs or hematopoietic stem cells.

Various BMP-4 sequences are found in the scientific literature; one example considered representative but not meant to be limiting in any way as to the scope of the invention or any claim is SEQ ID NO. 1, GenBank: AAC72278.1 (gi|3850195|bone morphogenetic protein-4 [Homo sapiens]): MIPGNRMLMVVLLCQVLLGGASHASLIPETGKKKVAEIQGHAGGRRSGQSHELL RDFEATLLQMFGLRRRPQPSKSAVIPDYMRDLYRLQSGEEEEEQIHSTGLEYPER PASRANTVRSFHHEEHLENIPGTSENSAFRFLFNLSSIPENEAISSAELRLFREQVD QGPDWERGFHRINIYEVMKPPAEVVPGHLITRLLDTRLVHHNVTRWETFDVSPA VLRWTREKQPNYGLAIEVTHLHQTRTHQGQHVRISRSLPQGSGNWAQLRPLLVT FGHDGRGHALTRRRRAKRSPKHHSQRARKKNKNCRRHSLYVDFSDVGWNDWI VAPPGYQAFYCHGDCPFPLADHLNSTNHAIVQTLVNSVNSSIPKACCVPTELSAIS MLYLDEYDKVVLKNYQEMVVEGCGCR (SEQ ID NO:01) having a length of408 amino acids.

The effectiveness of using a BMP-4 for such biasing toward a pre-adipocyte may be measured by various means known to those skilled in the art, such as but not limited to those described in Example 1 herein, which is incorporated into this section for its teachings.

A number of factors may influence the development and formation of adipocytes from pre-adipocytes. In various embodiments any combination of these may be employed to increase the percentage of pre-adipocytes that develop into adipocytes.

In some embodiments, a sample of living cells is obtained from a patient. The patient may be a human subject or an animal, such as a pet or livestock breed. The sample may be blood or adipose tissue. The cells so obtained may be segregated by a method known to those skilled in the art to obtain stem cells, and the cells (prior to or after the optional segregation) and/or their progeny are cultured in contact with a quantity of bone morphogenetic protein-4 for a suitable time and at a suitable temperature. Thereafter the resulting cells (optionally after further culturing, assessment, and/or segregation) which have pre-adipocyte characteristics are returned to the patient in a manner and location to achieve a therapeutic benefit.

Contacting a cell or plurality of cells with BMP-4 may be achieved by various approaches. A purified or crude solution of BMP-4 may be added at a desired concentration to a culture of target cells, such as MSCs. Alternatively, stem cells may be genetically modified to express, such as transiently, BMP-4. This may be via a vector, such as but not limited to a plasmid vector, or via transposition methods such as described in U.S. Pat. No. 8,192,934, issued to Savilahti et al. on Jun. 5, 2012, and incorporated by reference for its teachings of gene transfer methods. Recombination and/or other genetic techniques may be employed as are known in the art. Alternatively, an accessory cell that expresses BMP-4 may be added to a culture of target stem cells.

While the disclosure specifically describes use of mesenchymal stem cells as the initial source of cells, this is not meant to be limiting of the scope of the invention. For example, hematopoietic stem cells (HSCs), or other adult stem cells, may be utilized. Also, in various embodiments murine embryonic stem cells are excluded, particularly pluripotent stem cell lines such as those of C3H/10T½ are excluded from the scope of such embodiments. However, as noted above, in other embodiments embryonic stem cells may be manipulated to become MSCs before being subjected to BMP-4 according to the techniques described herein.

Pre-adipocyte cells and their progeny, obtained from the methods described herein using BMP-4 for differentiation/biasing from stem cells, may be used in a variety of applications, some requiring additional steps. Such pre-adipocyte cells may be used in scientific research. In some such instances the cells may comprise various genetic modifications such as to evaluate specific approaches and hypotheses, such as directed to congenital diseases, degenerative diseases, and/or diseases of social importance such as obesity and diabetes.

The cells obtained in accordance to the present disclosure may also be used in various compositions and methods to treat a subject. As one area of applications, pre-adipocyte cells may be used in surgical procedures, such as cosmetic surgical or other surgical procedures where it is desired to provide a composition that fills a space in or on the subject and where at least a portion of the composition comprises cells that could further differentiate to adipose cells or tissue.

Numerous such applications are envisioned. Specific examples are in cosmetic surgery where wrinkles, creases, depressions or voids (such as due to accidents, gun wounds, etc.) are in need of filling to provide a more normal and/or attractive appearance. The cells, and compositions comprising such cells, which may include cell and tissue lattices, and/or cytokines and other growth factors, and/or cytokine modulators and/or inhibitors, may be provided in kits, single-use volumes, and so forth, and may be used for the above applications as well as for tissue augmentation, such as breast augmentation and lipoinjection. Various surgical procedures which may employ cells and/or other compositions of the present invention are described in Grabb and Smith's Plastic Surgery Charles H. Thorne (Editor), Scott P. Bartlett (Editor), Robert W. Beasley (Editor), Sherrell J. Aston (Editor), Geoffrey C. Gurtner (Editor), Scott L. Spear (Editor), 6^{th} Edition (2006), ISBN-10: 0781746981, incorporated by reference for the descriptions of such surgical procedures.

As noted, compositions of the present invention may comprise cells of the present invention with other chemicals such as but not limited to cytokines and other growth factors, cytokine modulators and/or inhibitors, and/or matrices to help achieve a surgical and/or therapeutic result. For example, the cells of the present invention may promote angiogenesis, either with or without genetic modification to promote a production and/or secretion of an angiogenic chemical (whether a hormone, vascular growth factor, etc.). The cells, and/or compositions comprising the cells, may thereby promote angiogenesis when provided in a subject.

Compositions of the present invention may comprise lattices and other features, such as described in U.S. Pat. No. 4,505,266, entitled “Method of using a fibrous lattice” and issued Mar. 19, 1985, and U.S. Pat. No. 5,922,025, entitled “Soft Tissue Augmentation Material” and issued Jul. 13, 1999. These patents are incorporated by reference in their entireties, including the respective discussions of non-claimed previously known tissue lattice compositions and applications.

Numerous cytokines, growth factors, and cytokine modulators and inhibitors are known in the art. An exemplary listing of cytokines and growth factors is available at http://www.genscriptcom/cytokines_and_growth_factors.html?src=google&gclid=CPaQ rPvax7ACFQ9whwodqWy9Zg. Cytokines are described also in The Cytokine Handbook, Fourth Edition (2003), ISBN-10: 0126896631, Angus W. Thompson and Michael T. Lotze, editors, incorporated by reference in its entirety for teaching of cytokines. Any suitable combination of cytokines, growth factors, and cytokine modulators and inhibitors may be employed, in view of the teachings herein, in a composition comprising cells of described herein, optionally also with scaffolding material.

Genetic modification may be employed for some embodiments, such as for introducing a vector or other genetic construct comprising an oligonucleotide that expresses BMP-4 in a stem cell (and thereby supply BMP-4 to contact such cell). Nucleic acids and nucleic acid constructs, introduction of genetic material, and expression constructs (such as a vector) may be constructed and/or practiced using any number of techniques standard in the art. For example, chemical synthesis or recombinant techniques may be used. Such techniques as are described, for example, in Sambrook et al (Molecular Cloning: A laboratory manual, Cold Spring Harbor Laboratory Press, Cold Spring Harbor, N.Y., 1989), and more recent techniques are known to those skilled in the art and may be employed. Generally, the individual genes and regulatory elements will be operably linked to one another such that the genes can be expressed to thereby provide the desired protein(s). Suitable vectors for use in these embodiments will be appreciated by those of ordinary skill in the art. See for example, U.S. Patent Publications 20080233648; 20060134789; 20060188489; and US20060110440 are cited and incorporated herein for their teachings concerning the transfection of stem cells with sequences, and in particular sequences encoding a biasing factor.

The claims provided below are incorporated into the specification.

While a number of embodiments of the present invention have been shown and described herein in the present context, such embodiments are provided by way of example only, and not of limitation. Numerous variations, changes and substitutions will occur to those of skilled in the art without materially departing from the invention herein. For example, the present invention need not be limited to best mode disclosed herein, since other applications can equally benefit from the teachings of the present invention. Also, in the claims, any means-plus-function and step-plus-function clauses are intended to cover the structures and acts, respectively, described herein as performing the recited function and not only structural equivalents or act equivalents, but also equivalent structures or equivalent acts, respectively. Accordingly, all such modifications are intended to be included within the scope of this invention as defined in the following claims, in accordance with relevant law as to their interpretation.

### EXAMPLES

The following is presented as non-limiting of the full scope of the invention.

### Example 1

### Evaluation of BMP-4 for Biasing of Human Cells

The following was conducted to assess the effectiveness of bone morphogenic protein (BMP-4) on development of pre-adipocytes from human stem cells.

Methods

Cell Culture:

Human adipose tissue derived stem cells (ADSCs) were cultured in DMEM/F12 (Invitrogen) supplemented with 1% antibiotics (Gibco) and 10% FBS (Gibco). Cells were cultured in T-75 tissue culture treated flasks (Corning) and incubated at 37° C. in 5% CO2. Human MSCs were cultured in X-Vivo 15 (Lonza) with 5.0 U/mL Heparin (Sigma Aldrich) and 10% FBS (Gibco). Cells were cultured in T-75 tissue culture non-treated flasks (Corning) and incubated at 37° C. in 5% CO2. Cells were passaged into T-25 tissue culture treated flasks (Corning) prior to exposure to differentiation media.

Differentiation Media Exposure:

Cells were exposed to differentiation media when they reached a confluency of approximately 80%. To induce differentiation, cells were cultured in two differentiation medias, media A and media B. Media A was from a commercially available kit, StemPro® Adipogenesis Differentiation Kit (Invitrogen). Instructions regarding differentiation were followed according to the protocol provided by the kit. Media B was DMEM (Invitrogen) supplemented with 5 μg/mL antibiotics (Gibco), 10% mesenchymal FBS (Stem Cell Technologies), 2 mM L-glutamine (Gibco), and BMP-4 (Invitrogen). Cells were incubated at 37° C. in 5% CO2 for one week.

Oil Red O Staining:

The cells were fixed in 4% paraformaldehyde (Sigma) and stained according to company protocol (Electron Microscopy Solutions).

Immunocytochemistry:

Cells were fixed in 4% paraformaldehyde (Sigma) for 1 hour. Following fixation, cells were washed with 1×PBS (Lonza) then permealized with PBS-T (Lonza) containing 0.1% Triton-X (Fisher Scientific) for 10 minutes at room temperature. After a second wash in 1×PBS, cells were incubated in a blocking solution consisting of, 1% BSA (Fisher Scientific), 10% normal donkey serum (Jackson Immunology), 0.3M glycine (Sigma), 0.1% PBS-Tween for 1 hour. Cells were incubated in primary antibodies DLK-1 (ab89908) and AP-2 (ab76007) overnight at +4° C. at a concentration of 1:200. The following day samples were washed with 1×PBS and incubated in the dark with Alexa Fluor® 488 goat antimouse (DLK-1) and Alexa Fluor® 568 donkey antirabbit (AP2) at a 1:1000 dilution for 1 hour. Wash with 1×PBS. In order to visualize the cell nuclei Hoescht was used during the wash at a concentration of 1:10000.

Flow Cytometry Analysis:

Cells were lifted from the bottom of the flask using 0.25% Trypsin (Gibco) and 37° C. for 1 minute. Trypsin was the neutralized using serum containing media. To remove any excess media cells were washed with 1×PBS. Cells were fixed and permeabilized with methanol (EMD) at −20° C. for 15 minutes and were then blocked in BSA for 1 hour at room temperature. Cells were then incubated for 1 hour at room temperature with primary and secondary antibody: DLK-1 (ab89908)+FITC (Jackson Immunology); AP-2 (ab76007)+TRITC (Jackson Immunology); Alpha-Actin (Epitomics)+TRITC; CD34 (SantaCruz Bio)+FITC

Results

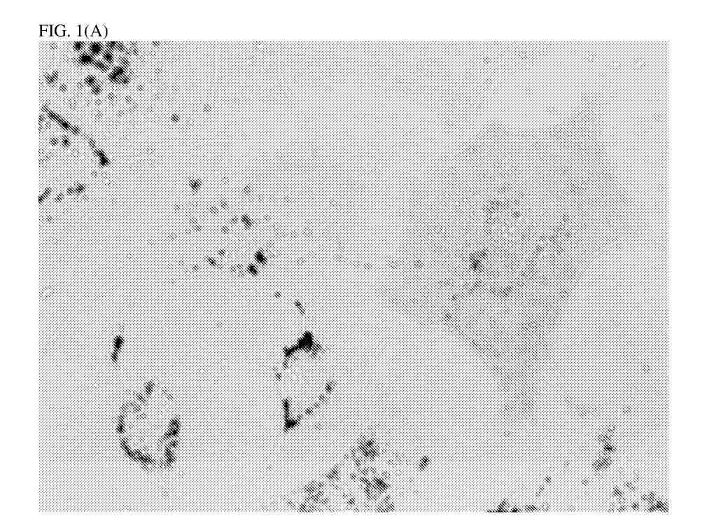

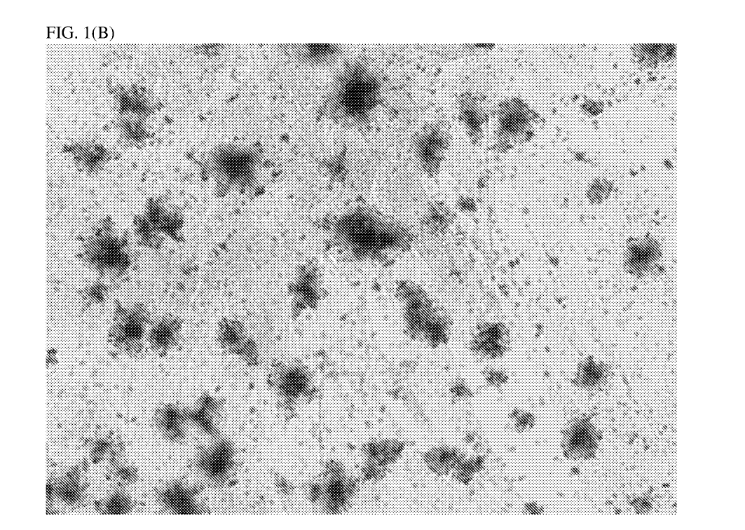

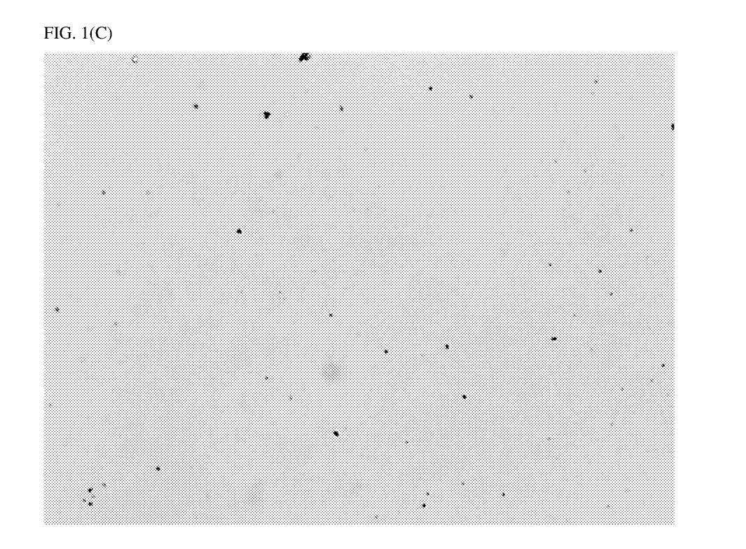

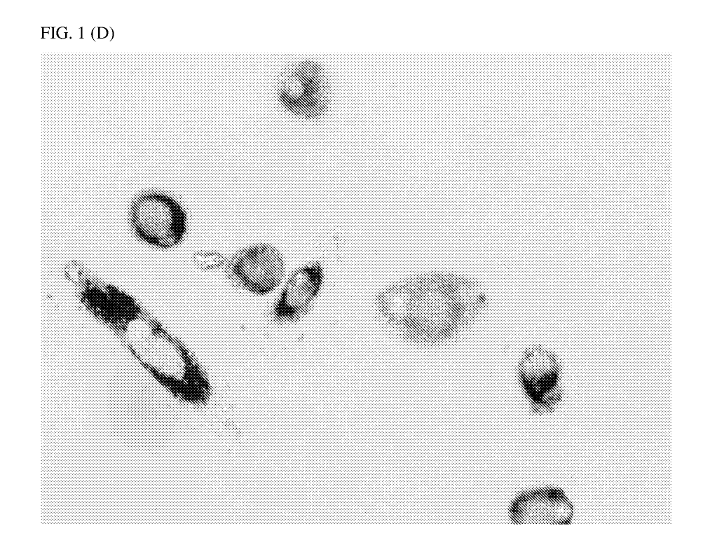

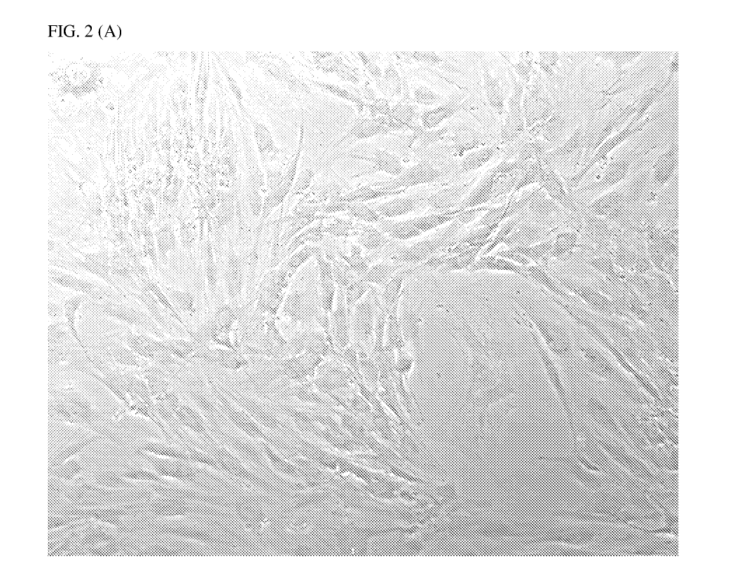

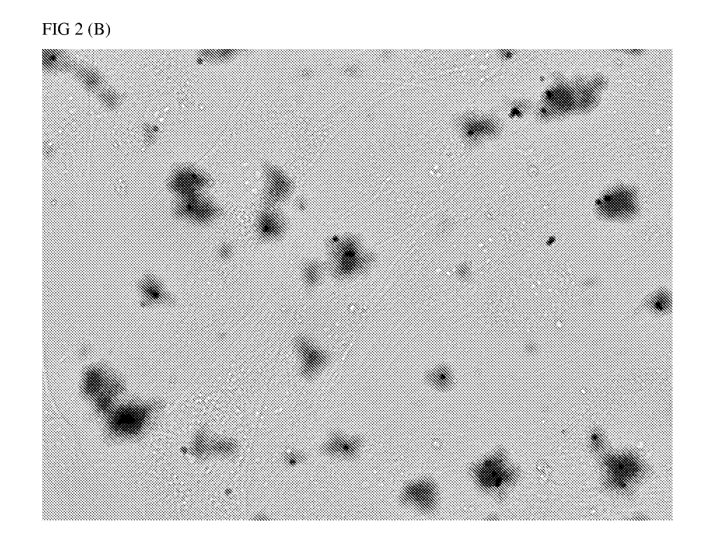

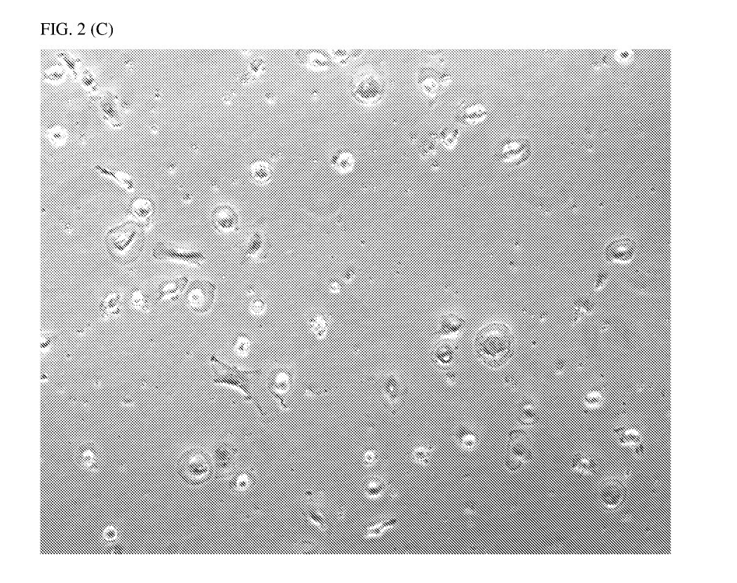

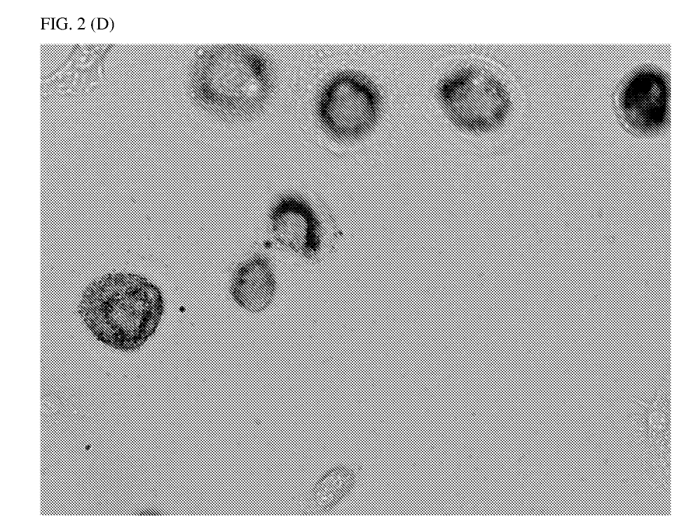

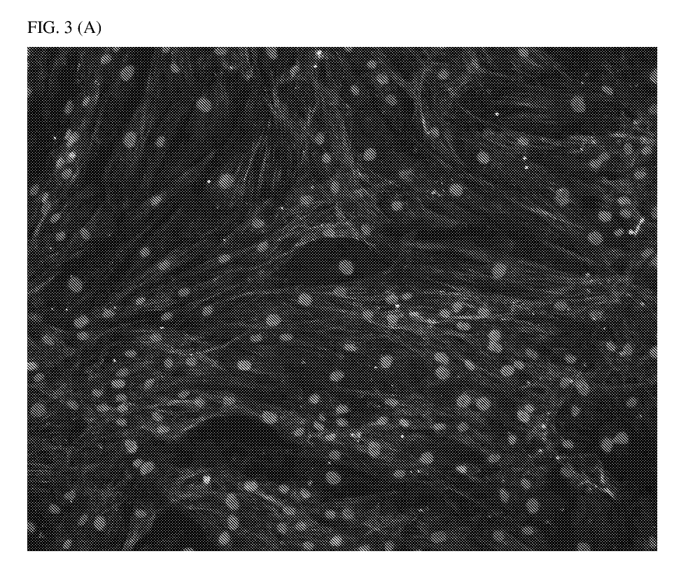

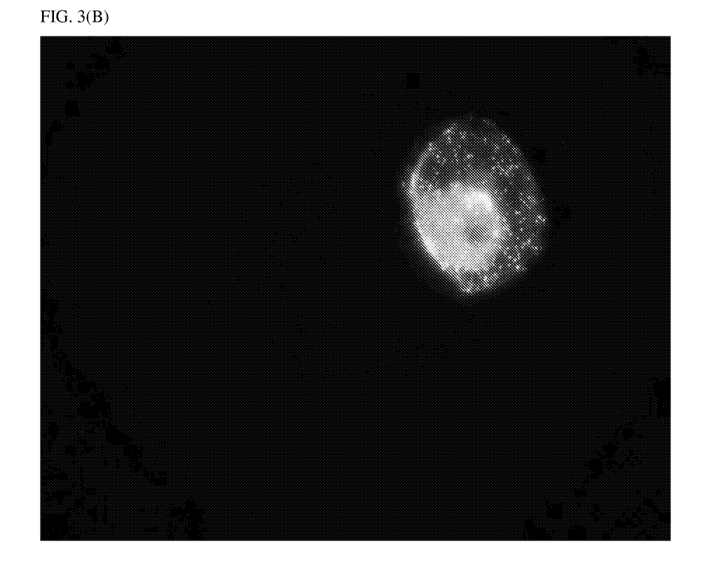

Treatment of MSCs and ADSCs with either the StemPro® Adipogenesis Differentiation Kit (Media A) or BMP-4 containing media (Media B) induced commitment to the adipose lineage. After one week of exposure to the differentiation medias cell morphology changes were apparent. The suspension cells (MSCs) began to adhere to the bottom of the flask. The ADSC culture (adherent) became more granular in appearance, as a consequence of the formation of triglyceride droplets. As demonstrated in FIG. 1 there are very low levels of commitment in the cells that had been differentiated using a commercially available kit compared to the cells that were differentiated in BMP-4. The formation of triglyceride droplets, within the cytoplasm, indicates the commitment to the adipocyte lineage (5). The formation of droplets was confirmed with Oil Red O staining, as seen in FIG. 2. Triglyceride droplet formation does not solely determine the formation of PAs, as adipocytes also form droplets. Immunocytochemistry was used to further verify the commitment to the adipose lineage by demonstrating the formation of PA and not adipocytes formation. Pre-adipocyte factor-1 (Pref-1), also known as DLK-1, is an inhibitor of adipogenesis and is expressed only in PAs at high levels (6). Fatty acid binding protein-4 (FABP-4), also known as AP2, is expressed in adipocytes and is not found in PAs (7). According to FIG. 3, PA formation was confirmed by positive staining for Pref-1 (green) and negative staining for FABP-4 (red).

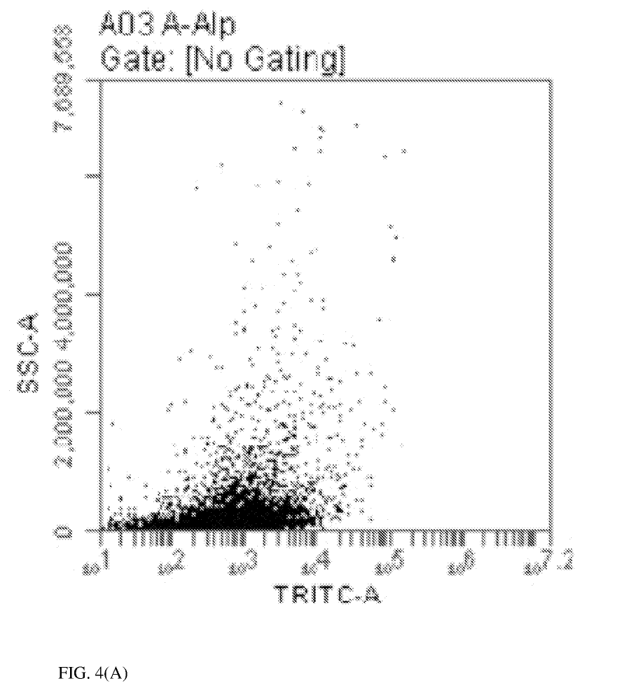

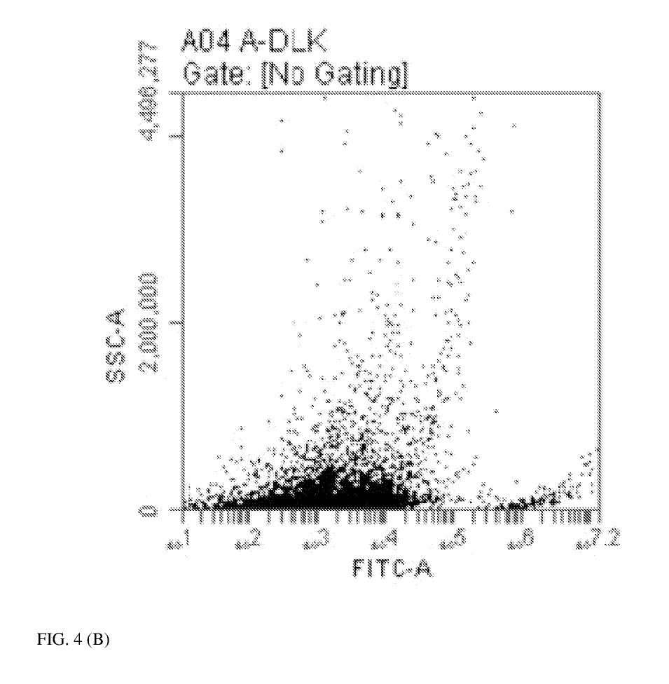

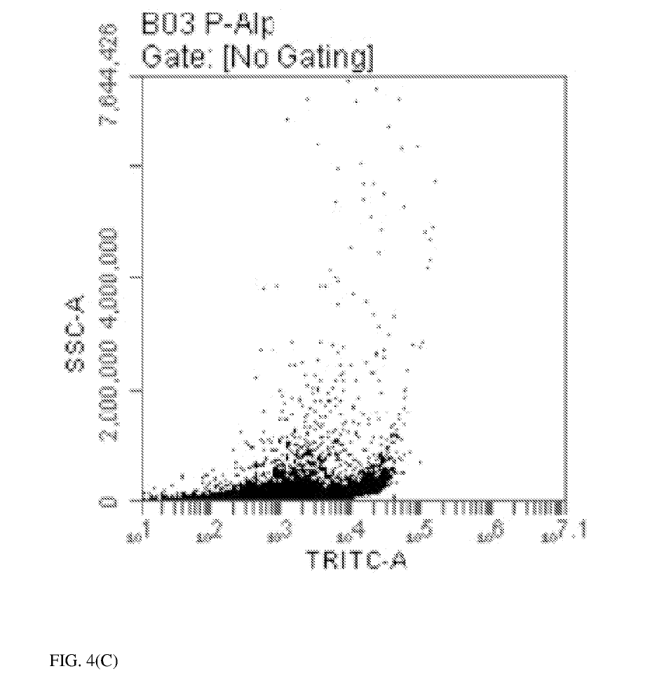

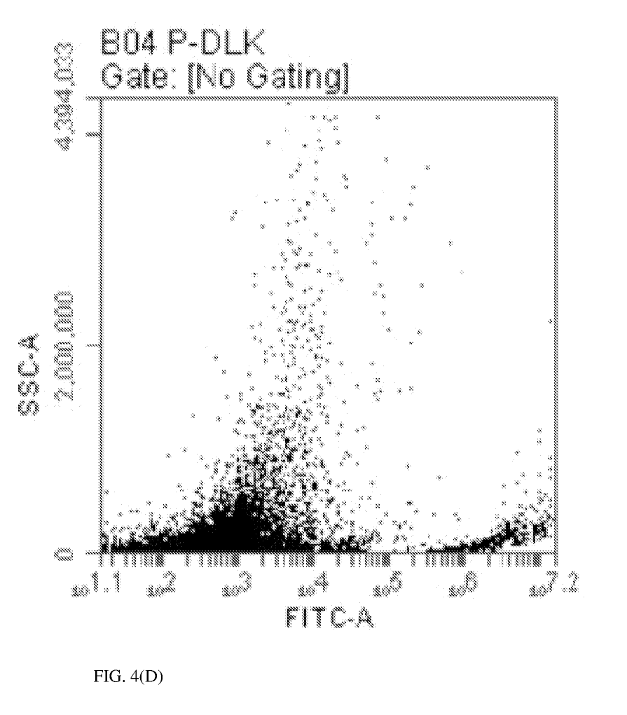

In order to look at the relative levels of expression, flow cytometry analysis was also performed. The cells were characterized using previously mentioned antibodies with the addition of α-actin as a control. α-actin is a known marker for smooth muscle and was selected as a control because MSCs may maintain the ability to form blood vessels, even under adipogenic conditions. Neither population of cells, MSCs or ADSCs, stained positive for the adipocyte marker AP2 (data not shown), indicating there was no formation of adipocytes. In both populations, cells demonstrated positive staining for Pref-1 (green), as displayed in FIG. 4. The ADSC population showed fewer Pref-1 positive cells than the MSC population. Furthermore the MSC population demonstrated positive staining for α-actin (red), whereas the ADSC population did not (FIG. 4). This indicates the MSCs maintain the ability to form smooth muscle and PAs simultaneously. The formation of smooth muscle in blood derived MSCs means that they may maintain the ability to become blood vessels. Based on this idea, cellular necrosis may be overcome by the simultaneous injection of PAs and blood vessel forming cells. Injection of more cells with the ability to repopulate and supply nutrients will create a fat store, replacing the dwindling population of adipocytes, and maintaining the area of fat augmentation.

Discussion:

The results of this experiment clearly indicate BMP-4 containing media effectively directs MSCs, derived from adipose tissue and peripheral blood, toward the adipose lineage. Findings suggest the benefit of BMP-4 containing media, over commercially available kits, is the increase in differentiation efficacy and the potential for clinical use. This study also indicates that media comprising BMP-4 directs cells to become PAs, committing to the adipose lineage, without terminally differentiating into adipocytes. In contrast commercially available kits do not appear able to discern between the two cell populations.

Furthermore, the location of harvesting the MSCs is very important to reduce patient exposure to invasive procedures. MSCs are most commonly obtained from bone marrow aspirates, an invasive and extremely painful procedure. Stem cells obtained from liposuction or a whole blood draw, would be a less invasive procedure, with the blood draw being the easiest and least invasive technique of the two.

Further, it is noted that current fat augmentation methods, such as autologous fat transfer procedures, are widely unpredictable. This is due to high levels of cellular necrosis and/or fat re-absorption at the site of injection (8). In an autologous fat transfer procedure, the number of terminally differentiated cells (adipocytes) totals more than the number of committed, yet undifferentiated cells (PAs). In this case, eventual cell loss is bound to occur, usually seen within six weeks (8). The natural process to regenerate fat involves replacing the lost adipocytes. This is more easily done when there is a population of progenitor cells. These are cells which are not fully differentiated but committed to becoming a specific type of cell (9). Injection of more cells with the ability to repopulate, such as PAs, will create a fat store, replacing the dwindling population of adipocytes, and maintaining the area of fat augmentation.

### REFERENCES

1. Pittenger, M., Mackay, A., Beck, S., Jaiswal, R., Douglas, R., Mosca, J., & . . . Marshak, D. Multilineage potential of adult human mesenchymal stem cells. Science, 284(5411): 143-147. 1999.
2. N. F. Pittenger, U.S. Pat. No. 5,827,740. 1998.
3. Tang, Q., Otto, T., and Lane D. Commitment of C3H10T½ pluripotent stem cells to the adipocyte lineage. Proceedings of the National Academy of Science. 101(6): 9607-11. 2004.
4. Cawthorn, W., Scheller, E., MacDougald, O. Adipose Tissue Stem Cells meet Preadipocyte Commitment: Going Back to the Future. The Journal of Lipid Research. 53(2):227-246. 2012.
5. Gregoire, F., Smas, C., and Sul, H. Understanding Adipocyte Differentiation. Physiol. Rev. 78: 783-809, 1998.
6. Wang, Y., Kim, K., Kim, J., and Sul, H. Pref-1, a Preadipocyte Secreted Factor That Inhibits Adipogenesis. The Journal of Nutrition: Recent Advances in Nutritional Sciences. 136: 2953-2956. 2006.
7. Urs, S., Smith, C., Campbel, B., Saton, A M., Taylor, J., Zhang, B., Snoddy, J., Jones Voy, B., Moustaid-Moussa, M. Gene expression profiling in human preadipocytes and adipocytes by microarray analysis. The Journal of Nutrition. 134(4): 762-70. 2004.
8. Ersek, R. Transplantation of purified Autologous Fat: A 3-year Followup is Disappointing. The Journal of Plastic Surgery. 87(2): 219-227.1991.

9. Yoshimura, K., Suga, H., Eto, H. Adipose-derived stem/progenitor cells: roles in adipose tissue remodeling and potential use for soft tissue augmentation. Regenerative Medicine. 4(2): 265-273. 2009.

It should be understood that the foregoing disclosure emphasizes certain specific embodiments of the invention and that all modifications or alternatives equivalent thereto are within the spirit and scope of the invention as set forth in the appended claims.

1. A method of biasing differentiation of a stem cell comprising: contacting a stem cell with bone morphogenetic protein-4 (BMP-4) for sufficient time and under conditions to bias the stem cell to a pre-adipoctye; and, obtaining the pre-adipocyte.

2. The method of claim 1 wherein the stem cell is a mesenchymal stem cell (MSC).

3. The method of claim 1 wherein a plurality of stem cells are contacted with the BMP-4, and a statistically meaningful percentage of the plurality become pre-adipocytes.

4. The method of claim 3 wherein the statistically meaningful percentage is at least 30, at least 40, at least 50, at least 60, at least 70, at least 80, or at least 90 percent.

5. The method of claim 1 wherein the contacting is achieved via genetically modifying the stem cell by providing an expressible oligonucleotide encoding BMP-4.

6. The method of claim 6 wherein the expressible oligonucleotide is under control of an inducible promoter.

7. A cell produced by the method of claim 1.

8. A method of modifying a stem cell comprising: contacting a stem cell with bone morphogenic protein-4 (BMP-4) under conditions to differentiate the stem cell (or its progeny) to a pre-adipocyte cell, and collecting the pre-adipocyte cell.

9. The method of claim 8, wherein the stem cell is a mesenchymal stem cell (MSC) or hematopoietic stem cell (HSC).

10. (canceled)

11. (canceled)

12. (canceled)

13. The method of claim 8, wherein the contacting comprises providing in the stem cell an oligonucleotide expressing BMP-4.

14. A cell produced by the method of claim 8.

15. A method of treating a subject comprising:
a. obtaining a sample of tissue from a subject that comprises stem cells;
b. contacting said stem cells with bone morphogenic protein-4 under conditions to obtain pre-adipocytes; and
c. delivering said pre-adipocytes to the subject to treat a condition requiring said pre-adipocytes.

16. The method of claim 15 wherein the subject is a human subject.

17. The method of claim 15 wherein the delivering comprises delivering said pre-adipocytes to a target of interest in the subject at which adipocyte deposition is required.

18. The method of claim 15, wherein the delivering comprises lipoinjection, reconstructive surgery, cosmetic surgery, tissue augmentation surgery.

19. (canceled)

20. (canceled)

21. (canceled)

22. The method of claim 1, wherein said stem cell is an adult stem cell.

23. The method of claim 1, wherein said contacting comprises subjecting said cell to culture media containing BMP-4 under conditions and for a period of time to induce differentiation of said cell to a pre-adipocyte.

24. The method of claim 15, wherein said sample of tissue is a blood sample, bone marrow sample, fat tissue, or muscle tissue.

25. The method of claim 8, wherein said contacting comprises subjecting said cell to culture media containing BMP-4 under conditions and for a period of time to induce differentiation of said cell to a pre-adipocyte.


SEQUENCE LISTING (1 sequence)

SEQ ID NO: 1 - protein, 408 aa, Homo sapiens
MIPGNRMLMVVLLCQVLLGGASHASLIPETGKKKVAEIQGHAGGRRSGQSHELLRDFEATLLQMFGLRRRPQPSKSAVIPDYMRDLYRLQSGEEEEEQIHSTGLEYPERPASRANTVRSFHHEEHLENIPGTSENSAFRFLFNLSSIPENEAISSAELRLFREQVDQGPDWERGFHRINIYEVMKPPAEVVPGHLITRLLDTRLVHHNVTRWETFDVSPAVLRWTREKQPNYGLAIEVTHLHQTRTHQGQHVRISRSLPQGSGNWAQLRPLLVTFGHDGRGHALTRRRRAKRSPKHHSQRARKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR